(19) United States
(12) Patent Application Publication
(10) Pub. No.: US 2009/0259288 A1
(43) Pub. Date: Oct. 15, 2009

(54) Catheter device for delivery of stents to bifurcated arteries

(76) Inventors: Bandula Wijay, Friendswood, TX (US); Nandhika Wijay, Friendswood, TX (US)

Correspondence Address: KAJANE MCMANUS, 550 MARINA PARKWAY, SUITE E2, PMB 88, CHULA VISTA, CA, 91910, US

(21) Appl. No.: 12/456,175

(22) Filed: Jun. 12, 2009

Related U.S. Application Data
(62) Division of application No. 11/139,729, filed on May 27, 2005.

(51) Int. Cl. A61F 2/06; A61M 29/00

(52) U.S. Cl. 623/1.11; 604/96.01

(57) ABSTRACT

The present invention uses several innovative means in order to place two stents into branched arteries that have atherosclerotic disease. The stent delivery catheter has means to place two separate guide wires into the two arteries, deploy the first stent in the main artery and provide an opening in the stent as well as a guide wire in the branch artery for the second stent carrying catheter to be threaded into the branch artery.

### CROSS REFERENCE TO RELATED APPLICATIONS

This application is a divisional of, and claims priority from, co-pending U.S. application Ser. No. 11/139,729, of the same title, filed on May 27, 2005.

### FIELD OF THE INVENTION

The present invention relates to a method and device for delivering specially designed stents to bifurcated arteries in the body cavity.

### BACKGROUND OF THE INVENTION

Stents have been in use to provide scaffolding to lesions that have undergone angioplasty. Angioplasty is a procedure by which narrowed arteries in the body, especially the coronary arteries, are dilated using a size limited inflatable balloon. After balloon inflation the artery can restenose due to tissue reaction to the balloon mediated injury. Stents, which are metallic and nonmetallic structures, are delivered crimped on angioplasty balloons to the lesion location and are expanded into position by the inflation of the angioplasty balloon. This procedure has become the first line of treatment for constricted arteries due to atherosclerotic disease. In the case of straight artery segments this procedure is relatively straightforward. Typically an angioplasty balloon carrying a stent is delivered to the lesion and deployed by balloon expansion. However when the lesion involves an area of a bifurcation in the artery, the procedure is considerably more complex for the following reasons. If a stent is positioned across a main branch of the artery then the stent will obstruct blood flow to the side branch artery. Furthermore, if the side branch of the artery also has atherosclerotic disease, then an operator must “open” struts of the first stent in order to place a second stent in the branch artery. It is very difficult or almost impossible for an operator to thread a guide wire, commonly used in catheterization procedures, across or between the struts of the stent first delivered. If however, one can develop means to place a stent in the main artery and also have the means to thread a guide wire across the struts of the stent first placed, so that a second balloon inflation and subsequent delivery of a second stent can be achieved, then such a procedure and the associated device will immensely improve the efficacy and the safety of bifurcated stenting procedure.

Arteries, especially coronary arteries are very small (2 mm-4 mm in internal diameter) and are angulated and tortuous in nature. As such, devices made for these arteries should preferably have a very low profile and be uniformly flexible so that the catheter can be advanced with ease and can cross the lesion with ease, in order to deliver the stent safely.

Several methods and devices have been developed and patented in the past by a number of developers and inventors. Review of the devices and the patent literature on the subject reveals that the devices previously invented are either very bulky and therefore cannot be threaded into coronary arteries or other small arteries in the body or are so cumbersome that the procedure will take considerable additional time to complete, thereby compromising the safety of the patient.

There are several factors for a successful stenting of an artery in an area of bifurcation. First, the first stent delivered should be delivered with proper orientation so that any opening provided in this stent is in alignment with the branched artery. Secondly, the operator should have means to thread a guide wire across or through an opening of the stent into the side branch of the artery. Thirdly, the opening should be large enough and free of any sharp edges so that a second catheter containing a second stent can be threaded through the opening easily without causing any damage to the second stent or the balloon carrying the second stent. Examples of prior art devices are as follows.

Richter in U.S. Pat. No. 6,770,092 B2 describes one such device where two balloons are provided side by side so that each balloon can carry a guide wire that can be threaded into each branch artery. The stent is placed over both balloons. While this method is probably workable, the fact that there are two balloons will make the device bulky. Also as both catheter tips end very close to each other, when a lesion is crossed the operator has to advance two catheter tips at the same time, which makes the procedure more difficult.

Richter in a second U.S. Pat. No. 6,770,091 B2 also describes a similar concept with two separate stents on the two balloons that are side by side. This design makes the device even more complex as the profile of the device, which now comprises balloons and stents, is even larger than the profile in the original device. Also the fact that a portion of the stent in the branch artery extends proximally into the main artery provides a stent having a figure “8” cross section, creating is a major obstruction to the flow of blood in the artery and is a significant disadvantage as well as a clot promoter. The apparatus described for the assembly of a stent inside coronary arteries is not a practical way to deploy stents to bifurcated arteries.

Chobotov et al. in U.S. Pat. No. 6,761,733 B2, describes a series of belts that are placed to constrict the endovascular device(s). These belts and the stents are released by manipulating a release wire, which is accessible from outside the body. Here again the device is bulky and complex. The addition of manipulation wires invariably will make the device stiffer and less tractable. This method also depends on the elastic recoil of the branch implant, which can be very complex, and the issue of how orientation is achieved for the delivery device is not addressed.

Chen et al. in U.S. Pat. No. 6,017,324 describes a device that has a bifurcated balloon apparently containing a bifurcated stent crimped on it. While this apparatus seems like an obvious solution to the problem, it is almost impossible to manufacture a low profile bifurcated balloon. Balloons are made from various polymers by a process of blow molding, previously extruded tubes making the balloon wall strong by bi-axially orienting the polymer molecules. While a dipping process can make a balloon that is bifurcated, such balloons do not possess the strength characteristics required to deploy stents. Furthermore, the presence of two balloon legs at the end of the catheter will cause the catheter to have a very large profile which will make it bulky and not tractable.

Wilson in U.S. Pat. No. 6,802,856 also describes a device similar to that of Chen above. Instead of a bifurcated balloon, Wilson provides two separate balloons with two separate guide wires. The stent is placed over both balloons. Here again the profile of the distal end of the catheter will be the sum of two profiles due to two balloons. In addition he describes the use of a sheath which also adds a “wall” and thereby increases the profile as well as the stiffness of the catheter.

Pazienza et al. in U.S. Pat. No. 6,802,859 B1 describes another bifurcated stent but fails to describe how the stent is delivered into a bifurcated artery. Also it is not disclosed how the stent can be oriented once it has been threaded into the vasculature.

Penn et al. in U.S. Pat. No. 6,811,566 B1 describes a bifurcated stent that has a side arm that will engage an branch artery. The side arm is connected to the main stent at about the middle of the main stent. Again this design is bulky due to the fact that both stents are joined and no workable description of alignment is described.

Vardi et al. in U.S. Pat. No. 6,835,203 B1 describes a stent and method of deployment. The stent assembly consists of an “independent tube”, which is placed outside the balloon but between the stent and the balloon. A guide wire is threaded into the side branch through this independent tube through a hole provided in a first stent that will be later deployed in the main artery. Once the wire is threaded a second stent is advanced over the wire to the side branch and deployed. The apparatus described, although solving some of the problems associated with orientation, is still a very bulky device that has a larger profile and is cumbersome to use.

Parodi U.S. Pat. No. 6,827,726 B2 describes an apparatus with multiple guide wires to canulate different arteries but the method works only with self expanding stents and again is very bulky when balloons are used for the deployment of the stents.

FIG. 5a is a prior art commercial design that uses two tubes 14 and 60 through which guidewires 8 and 17 are run through respectively. Catheter or tube 60 terminates part way along stent 5 so that guide wire 17 can make a lateral exit into side branch 3. This design makes the stent 5 asymmetrical because tube 60 is under it. The profile of the distal end of the assembly is increased because of the presence of tube 60 under stent 5. The distal end of the assembly in the region of stent 5 also becomes more stiff apart from being difficult to advance or rotate due to its asymmetrical profile.

For reasons set forth above it is believed that a specially designed device having a low profile (or same profile as a state of the art stent), that can reliably canulate the branch artery while canulating the main artery is needed. Such a device will work effectively and will reduce the time required to complete the procedure and therefore minimize injury due to excessive catheter manipulation, as would be the case when bulky devices are used. The present invention solves the above and also solves the problem of stent orientation while maintaining a low profile for the stent-balloon assembly, and by providing independent balloon inflation, fine rotational adjustment can also be accomplished during stent deployment. The catheter design is simple and can be easily manufactured by existing catheter technologies, making the device and the procedure cost effective.

The present invention is aimed at making a device that is quite simple to produce while effective for the intended purpose. The catheter device has a proximal end with facilities to introduce fluids for the inflation of the balloon for stent deployment and lumen(s) for introducing guide wire(s) that are threaded to the main and side branch arteries of the vasculature that is treated. The distal end of the catheter has tandem balloons that are either inflated together or separately, carrying the stent (crimped) over the tandem balloons. These balloons can be provided with a bulbous projection that is aligned with an exit opening for a second guide wire. The first guide wire (throughwire) exits the catheter at the very distal end of the catheter and is used to thread the catheter to the main artery while the second guide wire that exits between the two tandem balloons is used to thread the branched artery of the vasculature. In a typical procedure both guide wires are threaded into the catheter and approximately 20-30 cm of the guide wires is advanced beyond the catheter tip. The first guide wire is threaded into the main artery and the second guide wire is canulated and threaded into the branched artery. Once this is achieved the catheter is advanced over the guide wire slowly until the second wire exit port is aligned with the side branch of the vasculature. The operator can feel this when the catheter has reached the correct location, as the catheter will feel resistance to further advancement. In addition the operator can ensure this by injecting contrast dye under fluoroscopy and comparing the vascular anatomy to the location of the radiopaque band which is located between the tandem balloons. This will fix the position and orientation of the stent at the arterial junction. Once the position is confirmed, the distal balloon first and the proximal balloon next can be inflated to deploy the stent in the main artery. On the other hand both balloons can be inflated at the same time in order to deploy the stent. The first method described here has the advantage that the stent can be rotated slightly in order to make finer adjustments to the stent orientation. Once the stent is deployed the balloons are deflated and the catheter is slowly withdrawn while keeping both guide wires in palace. When the embodiment having the bulbous projections is used, the midsection of the stent having a special spiral window opens into a conical shape and forms a collar that will enter the branch artery ostium enabling easy passage for a subsequent catheter into the branch artery. A second catheter containing a stent-balloon is threaded over the guide wire that was threaded to the branch artery until the proximal end of the stent is in line with the edge of the inner diameter of the main artery. The balloon in the second catheter is then inflated to deploy the second stent such that the proximal end of the second stent is in touch with the side opening of the first stent. The second catheter is then withdrawn leaving the second stent in the side branch. If needed, the first or second balloon can be re-inserted to make any additional expansions in either stent in order to fully deploy the stent according to the current practice in stent deployment. The second catheter having a two stage balloon with its proximal balloon diameter relatively larger than the rest of the balloon will cause the stent opening to expand and take the shape of a conical collar that would fit to the shape of the branch artery ostium and or the protruding conical collar of the first stent. Post stent angiograms or intravascular ultrasound measurements are often performed to verify the accuracy of the stent deployment. Several designs are proposed in this application that achieve the same outcome. These are all variations of the same concept whereby two guide wires, which are either placed in the same lumen or separate lumens are used to advance one stent to the main artery and another stent to the branch artery. In the preferred embodiment one of the guide wires exits at or near the middle of the first stent. This is accomplished by using two tandem balloons, whereby the second guide wire exits between the balloons, that canulate the side branch artery during catheter advancement and positioning.

In either case the proposed novel concept is a major improvement to the state of the art as the proposed device maintains the same or similar profile of the stent-balloon combination as this design does not contain side by side balloon or side by side additional tube in order to carry the second guide wire, which therefore does not increase the overall profile of the catheter.

### SUMMARY OF THE INVENTION

The present invention discloses a delivery device and method for delivering stents to bifurcated arteries. The catheter device contains a distal angioplasty balloon segment onto which a stent to be delivered is crimped. The catheter device is advanced over a guide wire to the lesion location. The balloon segment is constructed from two balloons that are in tandem positions having an opening for a second guide wire to exit between the balloons. The balloons can be either inflated using two separate lumens so that they can be inflated independently or are inflated using one lumen so that both balloons can be inflated with, a single inflation. The balloons can also be designed with a bulbous projection that will cause the stent to open outwards producing a conical neck or collar that will engage with the ostium of the branch artery. The middle segment may contain a radiopaque marker so as to identify the exit location of the second guide wire. In either case two separate lumens may be provided for the two guide wires without sacrificing the profile of the balloon-stent segment of the delivery catheter device. This is achieved by providing two separate lumens for the two guide wires for the entire length of the catheter device except for a length at its distal region where the balloon is located, wherein both guide wires share the same lumen.

In another variation of the concept proposed the inner tube of the catheter device extends from the proximal end to the distal end and the balloon is assembled outside this tube. The balloon is inflated by passing fluid into an annular space between two co-axial lumens as is very traditional in angioplasty catheters. Two guide wires are advanced through the inner lumen with one exiting at the distal end and other guide wire exiting at a position midway in the balloon region and between the two tandem balloons. As described earlier, in this embodiment also, guide wires can be placed in two separate lumens to prevent the guide wires from wrapping around each other. The balloon is wrapped in a tri-fold (or multi-fold) configuration and the stent is crimped on to the folded balloon and a “guide wire threader” (not shown) is placed in the side hole so that the operator is able to remove the “guide wire threader” and pass the side branch guide wire into the lumen before the catheter device is introduced into guiding catheter device and hence the artery to be treated.

### BRIEF DESCRIPTION OF THE DRAWINGS

FIG. 1 shows a bifurcated artery requiring stenting both in the main artery and the branch artery.

FIG. 2 shows an ideal situation where one stent is placed in the main artery and another one in the branched artery.

FIG. 3 shows a stent being placed in the main artery using a PTCA balloon catheter.

FIG. 4 shows a stent placed in the main artery, which is blocking the blood flow to the branched artery.

FIG. 5 shows a stent delivery catheter device with the ability to deliver stents to bifurcated arteries.

FIG. 5a is a prior art design involving bifurcated arteries;

FIG. 6 is an enlarged view of an area between the tandem balloons of the device.

FIG. 7 shows an alternate embodiment of the stent delivery catheter device with special strategically located bulbous projections on the balloon, with the balloons expanded and the stent deployed, forming a collar which engages into the branched artery.

FIG. 8 shows the stent delivery catheter device in its initial configuration inside a bifurcated artery.

FIG. 9 shows the stent delivery catheter device with the balloons expanded and the stent deployed within the main branch of the bifurcated artery.

FIG. 10 shows a second stent delivery catheter device in the branched artery prior to the deployment of the stent.

FIG. 11 shows the stent delivery catheter device balloon inflated for second stent deployment

FIG. 12 shows an alternative embodiment for stent delivery catheter for the branched artery with the balloon having an expanded diameter at its proximal end.

FIG. 13 shows an alternate embodiment for delivering two stents to a bifurcated artery.

FIG. 14 shows cross section along lines 14-14 of FIG. 13 view of the embodiment of FIG. 13 at the double arrows showing the balloon deflated and containing a crimped on stent.

FIG. 15 shows another view of the embodiment of FIG. 13 in which the balloon is inflated, deploying the stent.

FIG. 16 is another cross section along lines 16-16 of FIG. 15 view of the embodiment of FIG. 13 showing the balloon inflated.

### DETAILED DESCRIPTION OF THE PREFERRED EMBODIMENTS

The object of this invention is to develop a simple and effective device for deploying stents in bifurcated arteries. The invention described herein is a device that comprises two or more angioplasty balloons mounted distally in tandem fashion on a catheter device shaft having facilities to place two or more guide wires. One guide wire is placed in a straight through lumen and a second guide wire is placed either in the same through lumen or a separate lumen, exiting in either case via an opening in the shaft between the two balloons.

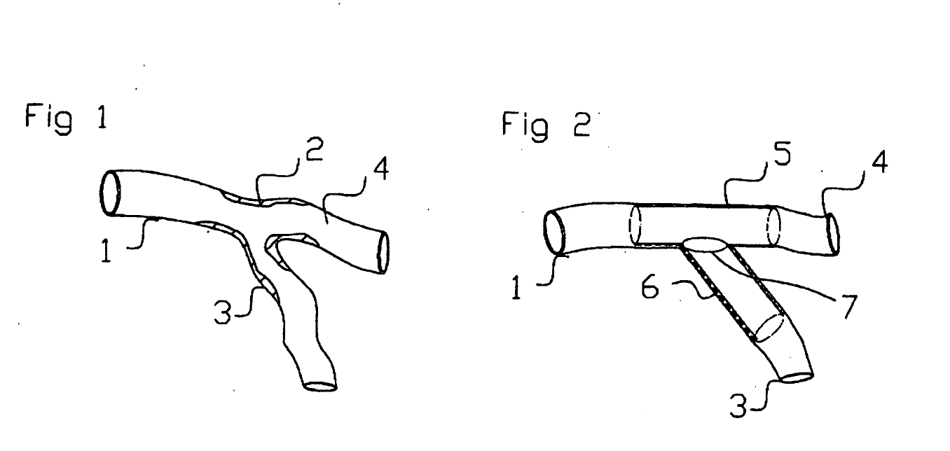

In FIG. 1 a bifurcated artery comprising a main branch 1 and a side branch 3, with a continuation 4 of the main branch 1 extending distally of the side branch 3. The bifurcated artery is seen to be atherosclerotic, having plaque 2 buildup therein.

In FIG. 2, ideal desired placement of stents, 5 in the main branch 1 and 4, and 6 in the side branch 3, is visualized, with an opening 7 formed in stent 5, allowing for unobstructed flow into stent 6.

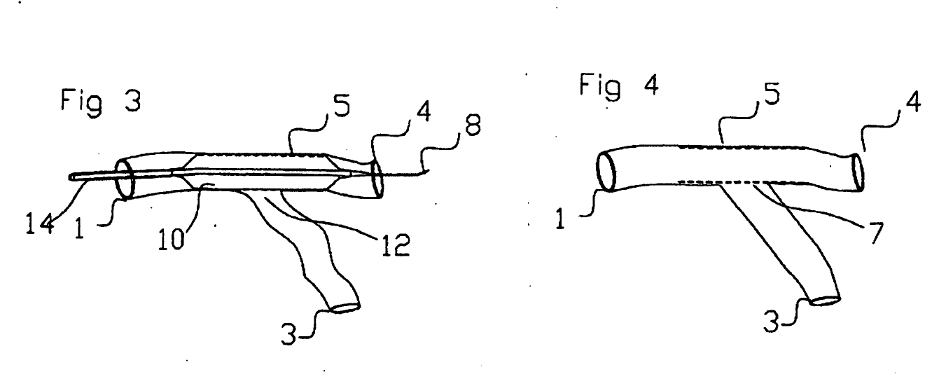

In FIG. 3, placement of stent 5 is illustrated. Here, guide wire 8 is placed into position in main branch 1 and 4, and stent 5, crimped (shown in the deployed state) onto balloon 10, is inserted over guide wire 8, to the desired position, and balloon 10 is inflated to expand stent 5 and then deflated and removed along with guide wire 8, leaving stent 5 in place as shown in FIG. 4.

Further to FIG. 4, the stent 5 is placed in the main artery 1 and 4, however the blood flow is blocked at position 7.

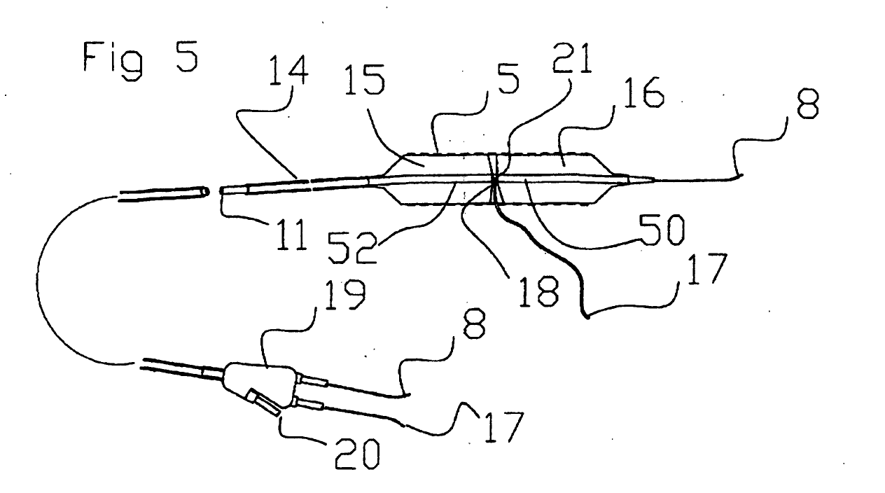

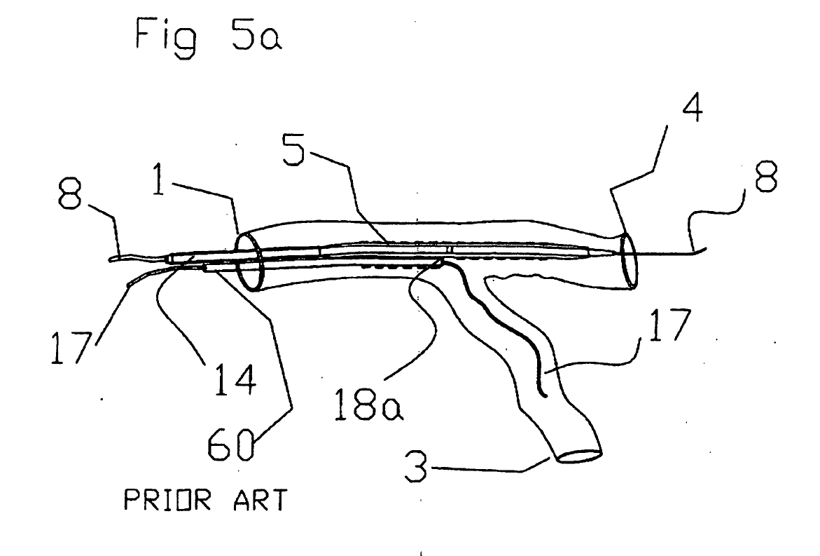

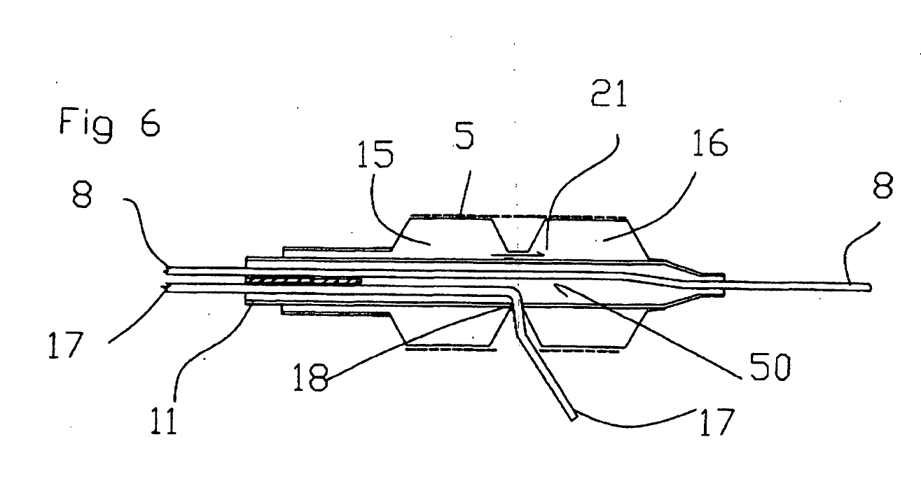

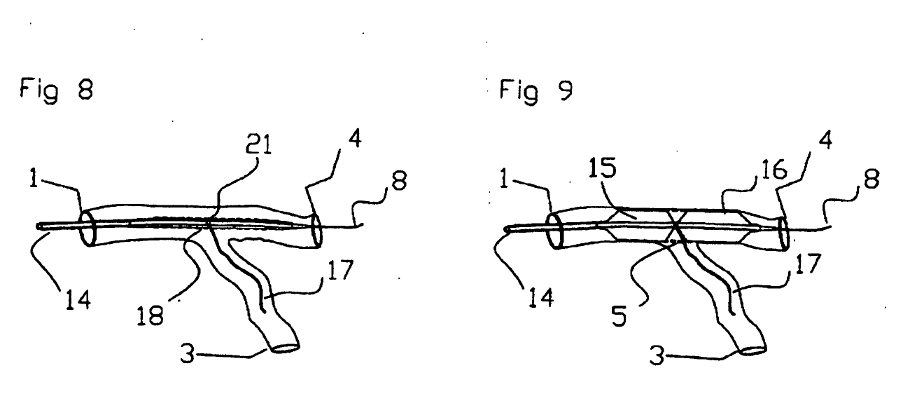

A first embodiment of such a device to facilitate the placement of two separate stents, easily and without compromising blood flow, in a bifurcated artery is achieved by the catheter device design shown in FIGS. 5-12. This device has a distal end 50 including two tandem balloons 15 and 16 mounted on a coaxial or bitumen catheter device shaft 14. The balloons 15, 16 can be inflated simultaneously by passing the inflating fluid via a lumen 20 (in hub 19) as in this design the two balloons communicate via a narrow opening as shown at 21 in FIG. 6 or separately by providing separate lumens for each balloon (not shown) in a proximal hub 19 of the device. The guide wire 8 also passes through catheter 14 in its central lumen 11. A side opening 18 between the balloons 15, 16, is provided for passage of a second guide wire 17 therethrough. The balloons 15 and 16 need not be the same size. It is preferred for balloon 16 to be smaller since the main branch 4 decreases in size after the side branch 3 juts off and balloon 15 is deployed in a vessel having a larger inside diameter. In this manner the balloon maximum diameters can be more closely tailored to the size of the vessel in which the stent 5 is to be set.

A radiopaque band or similar component 31 may be provided for use in locating the position of the guide wire opening 18 in relation to the arterial anatomy.

A wire form (not shown) is placed in the opening 18 to facilitate the operator in threading the guide wire 17 into the central lumen 11 of the device before the device is inserted into the body cavity. The inner diameter of distal end 50 of the central lumen 11 is designed to accommodate one guide wire 8 so as to maintain a low profile, while the portion 52 proximal to the opening 18 has an internal diameter that will accommodate both guide wires 8, 17. This concept will provide a low profile similar to a standard PTCA/stent catheter device at the very distal portion that enters the lesion, facilitating crossing of the lesion.

The catheter device 14 as described in the paragraph above is threaded over guide wires 8 and 17 using a guiding catheter commonly used for threading catheters to the coronary and other arteries of the body. The guide wire 8 is placed in the main artery 1, 4 and guide wire 17 is threaded and placed in the branch artery 3. The catheter is then slowly advanced such that the opening 18 is in line with the branch artery 3. The operator would feel this when the catheter couldn't be advanced any further.

Once the operator has located the side branch ostium as described above and also further confirmed by contrast injection and comparing the anatomy of the artery to the position of the radiopaque marker 31, the balloons 15 and 16 are inflated in order to deploy the stent 5 in the main artery 1, 4. The balloons 15 and 16 are then deflated and the shaft 14 is withdrawn leaving the guide wires 8 and 17 in place.

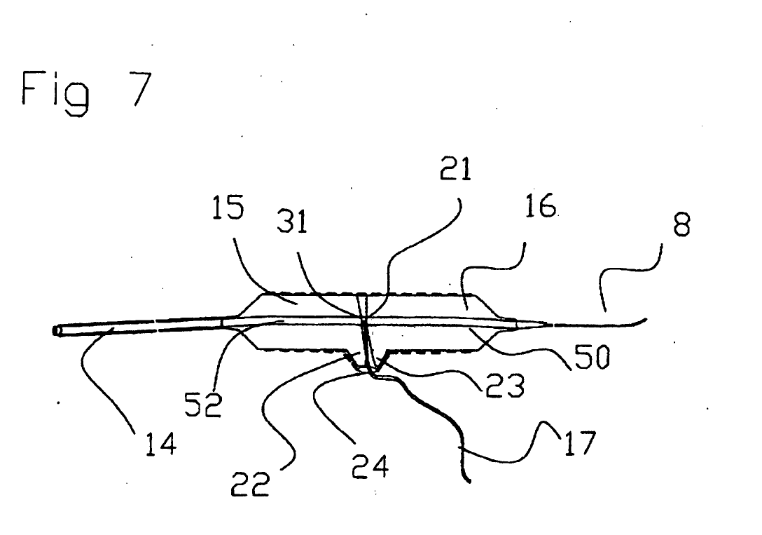

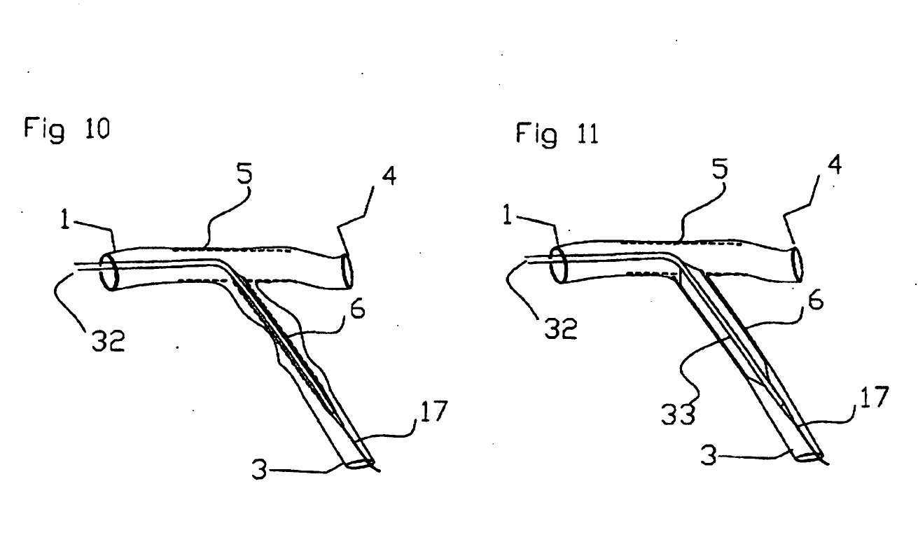

In FIG. 7, the balloons 15, 16 have a bulbous projection 22 and 23, respectively, located near the side opening 18 and oriented outwards on balloon inflation. When the two balloons 15 and 16 are then inflated, the bulbous projections 22 and 23 cause the stent 5 to expand unevenly, and form a conical collar that will engage with the ostium of the branch artery 3 and seating the stent 5 projection or conical collar into the branch artery ostium. This enables the threading of the second balloon catheter carrying the stent 6 to enter the branch artery 3 easier as shown in FIGS. 10 and 11. The conical collar is formed from the stent struts and have a spiral loops forming an opening for the passage of the guide wire 17. The spiral loops provide more strut support than strut loops that are generally radial forming an opening for the guide wire passage.

FIG. 10 illustrates the method for threading the second balloon catheter shaft 32 carrying the stent 6 over the guide wire 17 into the branched artery 3. The second catheter shaft 32 enters via the opening 7 in the stent 5 so formed by the bulbous projections 22 and 23 as previously described. However in the absence of the bulbous projections, it is still possible for the second catheter shaft 32 to be advanced through the opening 7 in the stent 5 into the branched artery 3.

Once the second balloon catheter shaft 32 is threaded into the branched artery 3, balloon 33 is inflated in order to deploy the stent 6 in the branch artery 3.

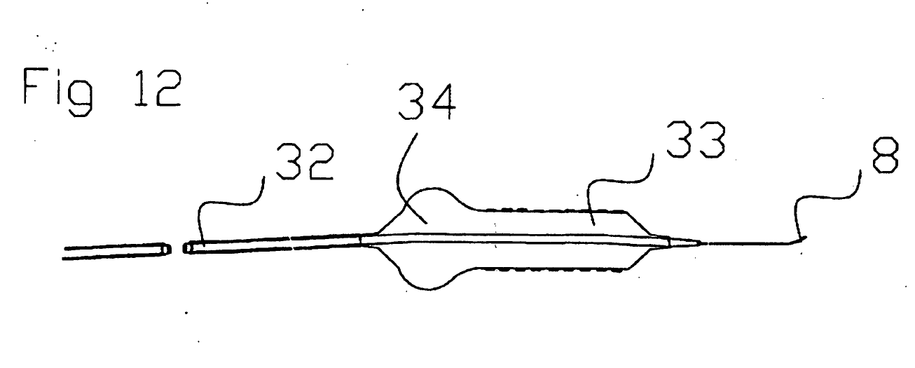

In an alternate embodiment for the second stent-balloon catheter for the branch artery as shown in FIG. 12 the second balloon 33 has a circular bulbous projection 34 that projects radially outward at a proximal end of the balloon 33. This enables the second catheter 32 to deform the opening 7 in the first stent 5 to form a conically shaped throat or collar that will engage and take the shape of the branch artery 3 ostium and mate with the first stent 5 without obstruction.

Once the second stent 6 is deployed, the balloon is deflated and the catheter is withdrawn. FIG. 2 shows the two stents 5, 6 deployed in a bifurcated artery deployed according to the present invention.

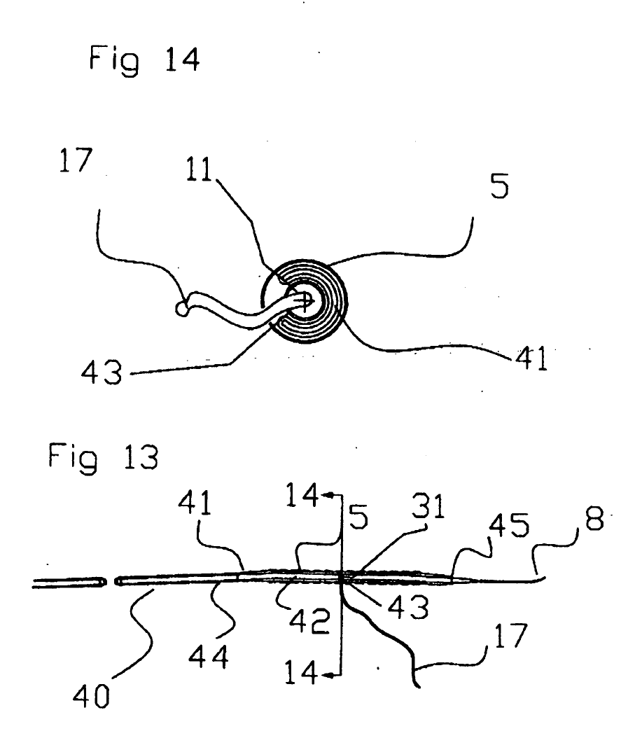

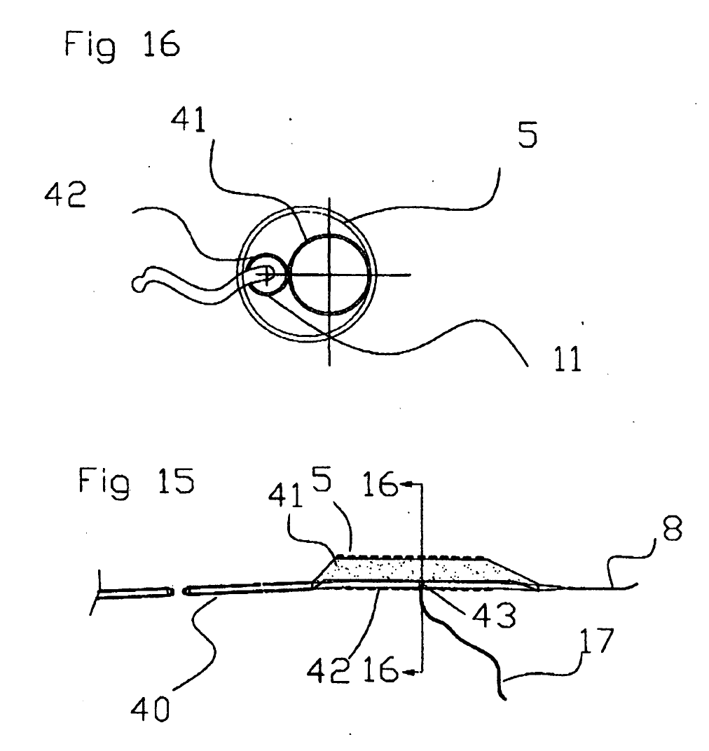

In yet another configuration, as shown in FIGS. 13-16, the balloon 41 in catheter 40 is situated outside of the central lumen (tube) 11. The balloon 41 is proximally attached to the catheter shaft 40 at 44 and distally to the catheter shaft 40 at 45. A side opening, preferably equidistant from the balloon proximal and distal end attachments, is provided on tube 11 at 43. The guide wire 17 can exit the catheter 40 through the side opening 43. A radiopaque marker 31 can again be provided at or near this location as described previously for improved fluoroscopic guidance.

The guide wire 8 exits the catheter 40 at the distal end as shown and previously described. It will of course be understood that the two guide wires 8 and 17 may be placed in two separate lumens or in one lumen or a combination thereof, in order to save space. The preferred embodiment should have two separate lumens for the two guide wires 8, 17 in most of the length of the catheter except under the balloon area or for a distance of one or two centimeters proximal to the proximal end of the balloon and extending to the tip of the catheter. This will help to prevent any tangling or self-winding of one guide wire on the other, while maintaining a low profile for the catheter, similar to a present day balloon catheters with a stent crimped onto it. The balloon 41 in this embodiment can be wrapped in known manner into a tri-fold (or multi fold) configuration and those who are familiar with the art will recognize various alternative means to provide a low profile for the wrapped balloon 41 containing the stent 5.

Once the two guide wires 8, 17 are placed in the two branches, one in the main 1, 4 and one in the branch 3, the catheter 40 is advanced as described until the operator feels resistance. The location is confirmed by contrast injection and the balloon 41 is inflated to deploy the stent 5. The balloon 41 is then deflated and withdrawn leaving the stent 5 and the guide wire 8 in the main artery and guide wire 17 in the branch artery 3, behind. A second balloon catheter 32 carrying stent 6 is threaded over the guide wire 17 as described and deployed as described before.

As previously noted, bulbous projection(s) 22 and 23 can be provided on the balloon 41 in this embodiment as well, however it should be noted that instead of two separate bulbous projections, it is also possible to provide one bulbous projection in this design to achieve the same objective, in order to force the side opening 7 in the stent 5 to open conically into the branch artery 3.

The foregoing disclosure and description of the invention are illustrative and explanatory thereof, and various changes in the size, shape and materials, as well as in the details of the illustrated construction, may be made without departing from the spirit of the invention.

We claim:

1. A catheter assembly for stenting a main and branch vessel, comprising:
an elongated body comprising at least one guide wire lumen and at least one balloon inflation lumen;
at least two spaced balloons located proximally and distally on said body and surrounding said guide wire lumen and allowing for a lateral exit from said guide wire lumen therebetween;
a first stent mounted over said balloons configured with a lateral opening to accept a guide wire that emerges from said lateral exit from said guide wire lumen;
at least one eccentric segment on at least one of said balloons and mounted adjacent to said lateral opening on said stent such that upon expansion of the stent by said balloons, which enlarges said lateral opening, said eccentric segment assists in retaining the enlarged dimension of said lateral opening.

2. A catheter assembly for stenting a main and branch vessel, comprising:
an elongated body comprising at least one guide wire lumen and at least one balloon inflation lumen;
at least two spaced balloons located proximally and distally on said body and surrounding said guide wire lumen and allowing for a lateral exit from said guide wire lumen therebetween;
a first stent mounted over said balloons configured with a lateral opening to accept a guide wire that emerges from said lateral exit from said guide wire lumen;
said first stent becomes asymmetrical adjacent said lateral opening thereof upon expansion to retain its orientation with said later opening aligned with the branch vessel.

3. A catheter assembly for stenting a main and branch vessel, comprising:
an elongated body comprising at least one guide wire lumen and at least one balloon inflation lumen;
at least two spaced balloons located proximally and distally on said body and surrounding said guide wire lumen and allowing for a lateral exit from said guide wire lumen therebetween;
a first stent mounted over said balloons configured with a lateral opening to accept a guide wire that emerges from said lateral exit from said guide wire lumen;
a second stent delivered through said lateral opening of said first stent for attachment thereto after expansion of said first stent;
said second stent delivered on a second elongated body and mounted to a second stent balloon thereon;
said second stent balloon comprising a proximal section extending beyond said second stent to enlarge said lateral opening in said first stent when expanding said second stent.

4. A catheter assembly for stenting a main and branch vessel, comprising:
an elongated body comprising a guide wire lumen and a balloon inflation lumen;
a balloon having a proximal and distal ends, said balloon inflation lumen terminating near said proximal end of said balloon, said guide wire lumen continuing beyond said distal end of said balloon and said guide wire lumen having a lateral exit beyond the end of said balloon inflation lumen.

5. The assembly of claim 4, wherein:
said balloon does not wrap around said guide wire lumen a full 360 degrees to allow said lateral exit to remain unobstructed upon balloon inflation.

6. The assembly of claim 5, wherein:
said lateral exit is positioned along the length of said balloon.

7. The assembly of claim 6, further comprising:
at least two guide wires in said guide wire lumen with a first extending to the distal end of said body and a second extending through said lateral exit.

8. The assembly of claim 7, further comprising:
a stent mounted over said balloon having a side opening to accept said second guide wire though it.

9. The assembly of claim 4, further comprising: said balloon has a larger dimension proximally of said lateral exit than distally.
